(19) United States
(12) Patent Application Publication
(10) Pub. No.: US 2017/0333477 A1
(43) Pub. Date: Nov. 23, 2017

(54) NOVEL FORMULATION OF PHYSIOLOGICAL CHITOSAN-INORGANIC SALT SOLUTION/BLOOD MIXTURES FOR TISSUE REPAIR

(71) Applicant: ORTHO REGENERATIVE TECHNOLOGIES INC., Kirkland (CA)

(72) Inventors: Wei Ouyang, Montreal (CA); Michael Buschmann, Montreal (CA); Caroline Hoemann, Montreal (CA); Marc Lavertu, Pointe-Claire (CA); Anik Chevrier, Point-Claire (CA)

(73) Assignee: ORTHO REGENERATIVE TECHNOLOGIES INC., Kirkland (CA)

(21) Appl. No.: 15/629,795

(22) Filed: Jun. 22, 2017

Related U.S. Application Data
(62) Division of application No. 13/510,661, filed on Sep. 4, 2012.
(60) Provisional application No. 61/262,786, filed on Nov. 19, 2009.

(51) Int. Cl. A61K 35/14; C08L 5/08; A61L 27/44; A61K 35/28; A61L 27/36; A61K 35/51; C08L 5/10; C08L 5/00

(52) U.S. Cl. CPC A61K 35/14; C08L 5/10; C08L 5/08; C08L 5/00; A61K 35/28; A61L 27/3616; A61K 35/51; A61L 27/44

(57) ABSTRACT

The present description relates to a polymer composition for use in repairing tissue of a patient comprising at least one blood component, as polymer, such a chitosan, and at least one inorganic salt, such as NaCl, method of the composition and method of preparing the composition.

### TECHNICAL FIELD

The present description relates to a novel composition comprising blood/chitosan-inorganic salt mixtures, wherein physiological chitosan-inorganic salt solutions mixed with blood solidify faster than chitosan-β glycerol-phosphate solutions with chitosans of specific molecular weights.

### BACKGROUND ART

Chitosan is a linear polysaccharide composed of β-(1-4)-linked D-glucosamine (deacetylated unit) and N-acetyl-D-glucosamine (acetylated unit), which primarily results from the alkaline deacetylation of chitin. Chitosan can exist in many structural conformations, depending on a variety of factors that include the degree of hydration, the electrolyte environment and the complexity of original chitin mixture. Chitosan and its amino-substituted derivatives are bioerodible, biocompatible and biodegradable cationic polymers that have been advanced for a wide variety of applications, including tissue engineering, drug and gene delivery, pharmaceutical formulation, scaffolds for cell growth and cell encapsulation, wound healing and surface hemostasis.

A well known property of chitosan is its solubility at acidic pH (<6) and insolubility at neutral pH, making its use in solution with living cells and tissues problematic. Various publications (Chenite, international patent application publication No. WO 99/07416; Chenite et al., 2000, Biomater., 21: 2155-2161; Chenite et al., 2001, Carbohyd. Polym., 46: 39-47) describe that admixing a polyol-phosphate dibasic salt, i.e. glycerol-phosphate (GP), to an aqueous solution of chitosan can increase the pH of the solution while avoiding precipitation of the polymer. In the presence of these particular salts, chitosan solutions of substantial concentration (0.5-3%) and high molecular weight (>several hundred kDa) remain liquid, at low or room temperature, for a long period of time with physiologically acceptable neutral pH region between 6.8 and 7.2. These chitosan-glycerol phosphate solutions which can gel upon mild heating (for example from 4 to 37° C.), are biocompatible, biodegradable and adhesive to human tissues, provide for new opportunities in the delivery of sensitive therapeutics.

Compositions containing blood and chitosan have been described previously (see for example U.S. Pat. No. 7,148,209, the content of which is enclosed herewith by reference). Chitosan is known for being a thrombogenic polymer (e.g. it can accelerate the coagulation of blood). Chitosan-GP solutions were combined with sheep peripheral whole blood to form a thrombogenic mixture that solidified and adhered to a full-thickness cartilage defect by using a sheep repair model. The obtained results showed that solidification of a chitosan-glycerol phosphate/blood implant in microfracture defects improved cartilage repair compared with microfracture alone by increasing the amount of tissue and improving its biochemical composition and cellular organization (Hoemann et al., 2005, J. Bone Joint Surg., 87A: 2671-2686). A bilateral rabbit cartilage repair model was developed to study the effect of chitosan-GP/blood implants on cartilage repair following marrow stimulation. Results showed that chitosan-GP structurally stabilized the blood clots by inhibiting the clot retraction. Treatment of drilled defect with chitosan-GP/blood clots led to the formation of more integrated and hyaline repair tissue above a more porous and vascularised subchondral bone plate compared to drilling alone (Hoemann et al., 2007, Osteoarthritis & Cartilage, 15: 78-89).

Chitosan-GP/blood implants also increase cell recruitment, transient vascularisation, subchondral remodeling and modulate inflammatory and repair cell phenotype suggesting that these events are in part responsible for increase quantity and quality of repair tissue zone (Chevrier et al., 2007, Osteoarthritis & Cartilage, 15: 316-327; Hoemann et al., 2010, Am. J. Sports Med., 38, 9: 1845-56). Ultrastructure and compositional detail of chitosan-GP/blood dots, chitosan-GP alone and clots containing whole blood only were investigated by environment scanning electron microscopy (ESEM) in conjunction with energy dispersive X-ray analysis (EDS) (Iliescu et al., 2007, Microsc. Res. Tech., 71: 236-247). It was shown that chitosan formed a network structure in both chitosan-GP gel and chitosan-GP/blood clots. However this structure was altered by aldehyde fixation to produce artifactual aggregates of chitosan microparticles. EDS analysis showed that the majority of glycerol phosphate can diffuse freely from chitosan-GP gel. Solidification mechanisms of chitosan-glycerol phosphate/blood implants were investigated as well. Results showed that chitosan-GP/blood implants solidify through coagulation mechanisms involving thrombin generation, platelet activation and fibrin polymerization. Clotting factors can be used to shorten the in situ solidification time of chitosan-GP/blood implants in microdrilled cartilage defects (Marchand et al., 2009. Osteoarthritis & Cartilage, 17: 953-960). However, to facilitate their use in the clinic, the coagulation time of blood/chitosan composition should be shortened

There is still a need for an improved blood/chitosan implant that will solidify faster than known chitosan-GP/blood implants solutions. It would be highly desirable to be provided with fast-coagulating blood/chitosan implants for tissue repair.

### SUMMARY

There is now provided polymer composition comprising a blood component, a polymer and an inorganic acid. Such polymer composition is useful in repairing a tissue in a subject

According to a first aspect, the present application provides a polymer composition comprising a blood component, a polymer and at least one inorganic salt as well as a polymer composition consisting essentially of a blood component, a polymer and at least one inorganic salt. In an embodiment, the polymer composition further comprises or contains a mineral acid or an organic acid, such as, for example, hydrochloric acid, nitric acid, phosphoric acid, sulfuric acid, boric acid, hydrofluoric acid and/or hydrobromic acid. In an embodiment, the polymer is chitosan, chitin, hyaluronan, glycosaminoglycan, chondroitin sulfate, keratin sulfate, dermatan sulfate, heparin and/or heparin sulfate. In another embodiment, the inorganic salt is sodium salt, chloride salt, potassium salt, calcium salt, magnesium salt, phosphate salt, sulfate salt and/or carboxylate salt. In yet a further embodiment, the at least one inorganic salt is NaCl, KCl, CsCl, CaCl₂, CsF, KClO₄, NaNO₃ and/or CaSO₄. In still another embodiment, the blood component is whole blood, processed blood, venous blood, arterial blood, blood from bone, blood from bone-marrow, bone marrow, umbilical cord blood and/or placenta blood. In still a further embodiment, the composition is a gel. In another embodiment, the blood component is coagulated. In another embodiment, the polymer composition has a pH between 6.0 and 7.8, between 6.2 and 6.7 or of 6.6. In yet another embodiment, the polymer composition has an osmolality between 200 mOsm/kg and 600 mOsm/kg, between 326 mOsm/kg and 356 mOsm/kg, or of 354 mOsm/kg. In still another embodiment, the polymer is chitosan with a degree of deacetylation (DDA) between 20% to 100% or between 76% and 98%. In another embodiment, the polymer is chitosan with a number average molecular weight (Mₙ) between 1 kDa to 10 MDa or between 2.7 kDa and 298 kDa. In still another embodiment, the blood component: polymer ratio is 3:1 (vlv).

According to a second aspect, the present application provides a polymer composition comprising chitosan, hydrochloric acid, NaCl and a blood component and a polymer composition consisting essentially of chitosan, hydrochloric acid, NaCl and a blood component. In an embodiment, the polymer composition is prepared with a chitosan solution having a chitosan concentration of about 1.62% w/w. In another embodiment, the polymer composition is prepared with a hydrochloric acid solution having a hydrochloric concentration of about 38 mM. In still another embodiment, the polymer composition is prepared with a NaCl solution having a NaCl concentration of about 160 mM. Various embodiments with respect to the polymer composition pH, osmolality have been presented and do apply herein. Various embodiments of the chitosan degree and molecular weight have been presented and do apply herein. Various embodiment of the blood component have been presented and do apply herein.

According to a third aspect, the present application provides a method for repairing a tissue in a subject in need thereof, said method comprising the step of introducing into said tissue a polymer composition as defined herein such that the polymer composition adheres to the tissue and promotes cell proliferation for repairing the tissue. In an embodiment, the tissue is selected from the group consisting of cartilage, meniscus, ligament, tendon, bone, skin, comea, periodontal tissues, maxillofacial tissues, temporomandibular tissues, abscesses, resected tumors and ulcers.

According to a fourth aspect, the present application provides the use of a polymer composition as defined herein for repairing a tissue of a subject, wherein the polymer composition adheres to the tissue and promotes cell proliferation for repairing the tissue as well as the use of a polymer composition as defined herein in the manufacture of a medicament for repairing a tissue of a subject, wherein the polymer composition adheres to the tissue and promotes cell proliferation. In an embodiment, the tissue is selected from the group consisting of cartilage, meniscus, ligament, tendon, bone, skin, comea, periodontal tissues, maxillofacial tissues, temporomandibular tissues, abscesses, resected tumors and ulcers.

According to a fifth aspect, the present application provides a polymer composition as defined herein for repairing a tissue in a subject, wherein the polymer composition adheres to the tissue and promotes cell proliferation. In an embodiment, the tissue is selected from the group consisting of cartilage, meniscus, ligament, tendon, bone, skin, cornea, periodontal tissues, maxillofacial tissues, temporomandibular tissues, abscesses, resected tumors and ulcers.

According to a sixth aspect, the present application provides a method of preparing a polymer composition for repairing a tissue in a subject. Broadly, the method comprises (or consists essentially of) the step of: dissolving chitosan in HCl to provide a chitosan-HCl mixture; adding a NaCl solution to the chitosan-HCl mixture to provide a chitosan-HCl—NaCl mixture; and admixing at least one blood component to the chitosan-HCl—NaCl mixture to provide the polymer composition. In an embodiment, the chitosan is dissolved in HCl by heating at a temperature of 60° C. In a further embodiment, the concentration of chitosan in the chitosan-HCl—NaCl mixture is about 1.62% w/w of chitosan, the concentration of hydrochloric acid in the chitosan-HCl—NaCl mixture is about 38 mM of hydrochloric acid and/or the concentration of NaCl in the chitosan-HCl—NaCl mixture is about 160 mM Various embodiments of the blood component, the pH of the polymer composition the osmolality of the polymer composition, the polymer, the chitosan and the inorganic salt have been described and do apply herein.

In a particular embodiment, the polymer composition comprises 1.62% w/w of chitosan, 38 mM of hydrochloric acid and 160 mM of NaCl mixed with blood.

It is also provided herein a method for repairing a tissue of a patient, the method comprising the step of introducing into the tissue a polymer gel composition as defined herein such that the composition adheres to the tissue and promotes cell recruitment and other events for repairing the tissue. The composition can be placed or injected into a body site where the mixture aids the tissue repair, regeneration and reconstruction.

In an embodiment, the tissue is selected from the group consisting of cartilage, meniscus, ligament, tendon, bone, skin, cornea, periodontal tissues, maxillofacial tissues, temporomandibular tissues, abscesses, resected tumors, and ulcers.

It is also provided herein a method of preparing a polymer composition for repairing tissues in a subject, the method comprising the steps of dissolving chitosan in HCl; adding a NaCl solution to the chitosan-HCl mixture; and admixing at least one blood component to the chitosan-HCl—NaCl.

In an additional embodiment, the chitosan is dissolved in HCl by heating at 60° C. for about 2 hours.

It is also provided herein the use of the composition as defined herein for repairing a tissue of a patient and/or in the manufacture of a medicament for repairing a tissue of a patient, wherein the composition adhere to the tissue and promote cell proliferation for repairing the tissue.

### BRIEF DESCRIPTION OF THE DRAWINGS

Reference will now be made to the accompanying drawings.

FIG. 1 is a histogram of the coagulation time of blood/chitosan clots, prepared with chitosan number average molecular weight (Mₙ) of 75 kDa; 79% DDA, showing that all the blood/chitosan mixtures coagulated within 15 minutesand that blood/chitosan-HCl—NaCl mixtures coagulated faster than blood/chitosan-HCl-βGP (β Glycerol Phophate) mixtures in 3 of 4 rabbits.

FIG. 2 is a histogram of the mechanical strength of blood/chitosan-HCl—NaCl and blood/chitosan-HCl-βGP clots prepared with chitosan Mₙ 75 kDa; 79% DDA with blood from four different rabbits.

FIG. 3 is a histogram of the coagulation time of blood/chitosan clots, prepared with chitosan Mₙ 2.7 kDa; 98% DDA, showing that all the blood/chitosan mixtures coagulated within 18 minutes and that blood/chitosan-HCl—NaCl mixtures clotted faster than blood/chitosan-HCl-PGP mixtures.

FIG. 4 is a histogram of the mechanical strength of blood/chitosan-HCl—NaCl and blood/chitosan-HCl-pGP clots prepared with chitosan Mₙ 2.7 kDa; 98% DDA in triplicate.

FIG. 5 is a histogram of the coagulation time of blood/chitosan clots, prepared with chitosan Mₙ 75 kDa; 79% DDA, showing that all the blood/chitosan mixtures coagulated within 13 minutes and that blood/chitosan-HCl—NaCl mixtures clotted faster than blood/chitosan-HCl-βGP mixtures.

FIG. 6 is a histogram of the mechanical strength of blood/chitosan-HCl—NaCl and blood/chitosan-HCl-βGP clots prepared with chitosan Mₙ 75 kDa; 79% DDA in triplicate.

FIG. 7 is a histogram of the coagulation time of blood/chitosan clots, prepared with chitosan Mₙ 232 kDa; 81% DDA, showing that all the blood/chitosan-HCl—NaCl and blood/chitosan-HCl-βGP mixtures coagulated within 7 minutes.

FIG. 8 is a histogram of the mechanical strength of blood/chitosan-HCl—NaCl and blood/chitosan-HCl-βGP clots prepared with chitosan Mₙ 232 kDa; 81% DDA in triplicate.

FIG. 9 is a histogram of the coagulation time of blood/chitosan clots, prepared with chitosan Mₙ 298 kDa; 76% DDA, showing that all the blood/chitosan-HCl—NaCl and blood/chitosan-HCl-βGP mixtures coagulated within 7 minutes.

FIG. 10 is a histogram of the mechanical strength of blood/chitosan-HCl—NaCl and blood/chitosan-HCl-βGP clots prepared with chitosan Mₙ 298 kDa; 76% DDA in triplicate.

### DETAILED DESCRIPTION

It is provided a novel polymer composition comprising a blood component, a polymer and at least one inorganic salt. Related uses and methods are also provided. In an embodiment, such composition can be used for repairing a tissue in a subject. In yet a further embodiment, such composition can be used to limit the time associated with the coagulation of blood in a chitosan composition.

The polymer composition comprises a polymer, such as, for example chitosan, chitin, hyaluronan, glycosaminoglycan, chondroitin sulfate, keratin sulfate, dermatan sulfate, heparin and heparin sulfate. The polymer should be able to form a gel and be used for the treatment of tissue repair.

The polymer composition also comprises a blood component. Any blood component is contemplated herein, such as whole blood, processed blood, venous blood, arterial blood, blood from bone, blood from bone-marrow, bone marrow, umbilical cord blood or placenta blood. In an embodiment, the blood component is derived from whole blood to be enriched or depleted for a specific blood component. In a preferred embodiment, the mix ratio between the blood component and the polymer is about 3:1

The polymer composition also comprises at least one inorganic salt. In a preferred embodiment, NaCl is used. Alternatively, any inorganic salt, including sodium, chloride, potassium, calcium, magnesium, phosphate, sulfate, carboxylate salt, such as KCl, CsCl, CaCl₂, CsF, KClO₄, NaNO₃ and CaSO₄ are also contemplated herein.

The polymer composition can further comprise an acid, such as a mineral acid or an organic acid. The acid is used to lower the pH of the composition to facilitate dissolution of the chitosan. In a preferred embodiment, HCl is used as the acid. Alternatively, acetic acid, nitric acid, phosphoric acid, sulfuric acid, boric acid, hydrofluoric acid or hydrobromic acid could also be used.

In another embodiment, the polymer composition consists essentially of a blood component, a polymer and at least one inorganic salt. Such polymer composition does not contain additional components which participates to the formation of the gel or coagulation of the blood (such as coagulation products for example) but can contain other components such as an acid (to facilitate the dissolution of the chitosan), preservatives, etc.

It is specifically described herein the formulation of chitosan and a blood component, under physiological conditions (pH, osmolality). Briefly chitosan-HCl—NaCl solutions are mixed with blood, to create blood/chitosan-HCl—NaCl. The blood/chitosan-HCl—NaCl compositions are capable of forming a gel. In addition, the blood component in the blood/chitosan-HCl—NaCl is capable of coagulating to form an implant for tissue repair. In addition, when chitosan having a number average molecular weight lower than Mₙ 232 kDa is used, the chitosan-HCl—NaCl solutions mixed with blood described herein coagulates faster than chitosan-HCl-βGP solutions.

A preferred embodiment is shown in Example 1 where a novel formulation of physiological chitosan-HCl—NaCl solution was prepared with chitosan Mₙ 75 kDa; 79% DDA and mixed with whole blood. Blood/chitosan-HCl—NaCl mixtures coagulated faster than blood/chitosan-HCl-βGP mixtures to create fast-coagulating blood/chitosan implants for tissue repair.

Chitosan (1.62% w/w)-HCl (38 mM)-NaCl (160 mM) solution at pH of 6.50 and osmolality of 354 mOsm/kg, and chitosan (1.62% w/w)-HCl (71 mM)-PGP (2.15% w/w) solution at pH of 6.65 and osmolality of 334 mOsm/kg (Table 1) were mixed with fresh rabbit whole blood. To prepare chitosan/blood clots, immediately following collection of whole blood, a volume of 900 μl of whole blood was mixed into a cryotube containing 300 μl of 1.62% chitosan-HCl—NaCl solution (or 1.62% chitosan-HCl-βGP solution) and three 0.39 g stainless steel balls. The mixture was shaken by hand for 10 seconds (the mix ratio of blood to chitosan was 3 to 1).

250 μl was transferred into 3 glass tubes at 37° C. with a 1 ml syringe to prepare 3 clots (implants). One clot was used to test coagulation time and fixed immediately after it coagulated, the second clot was used to test coagulation time and fixed at 60 minutes after mixing, and the third clot was used to test coagulation time and mechanical strength after 60 minutes.

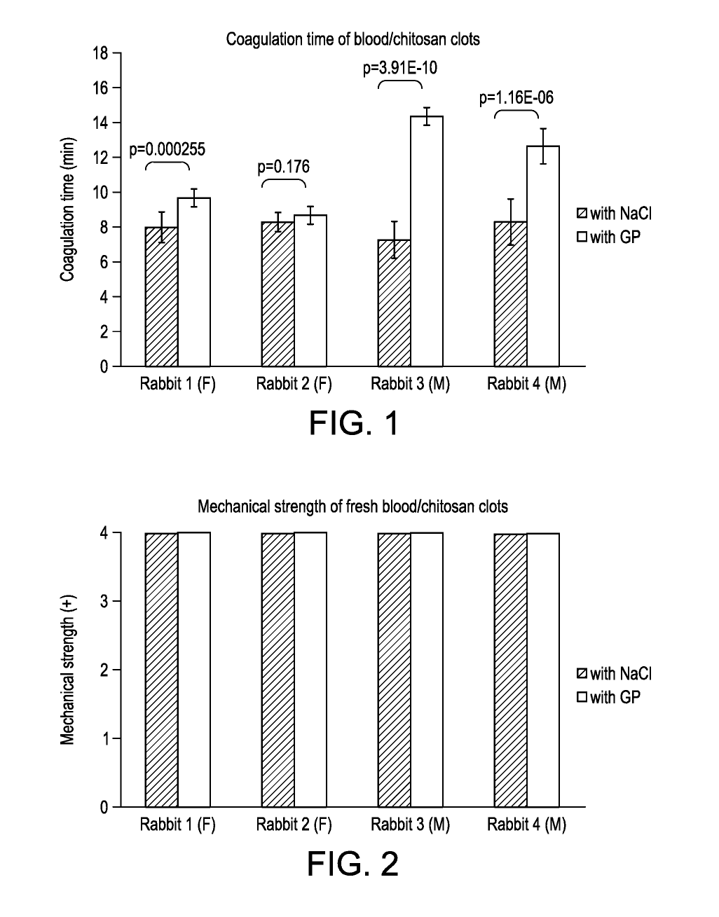

Coagulation time results (Tables 2-5 and FIG. 1) showed that all blood/chitosan mixtures prepared with chitosan Mₙ 75 kDa; 79% DDA coagulated within 15 minutes (from 6 minutes to 15 minutes). In 3 of 4 rabbits, the coagulation time of the blood/chitosan-HCl—NaCl mixtures was less than the coagulation time of the blood/chitosan-HCl-βGP mixtures (p<0.05). In the best case, coagulation time was shortened by a factor of 2. The mechanical strength results (Table 6 and FIG. 2) demonstrated that all the fresh blood/chitosan mixture clots were firm and elastic gel, the mechanical strength of all the clots was scored as “++++”.

After 60 minutes, some serum exuded from the clots (Table 6). The homogeneity evaluation results are shown in Table 7.

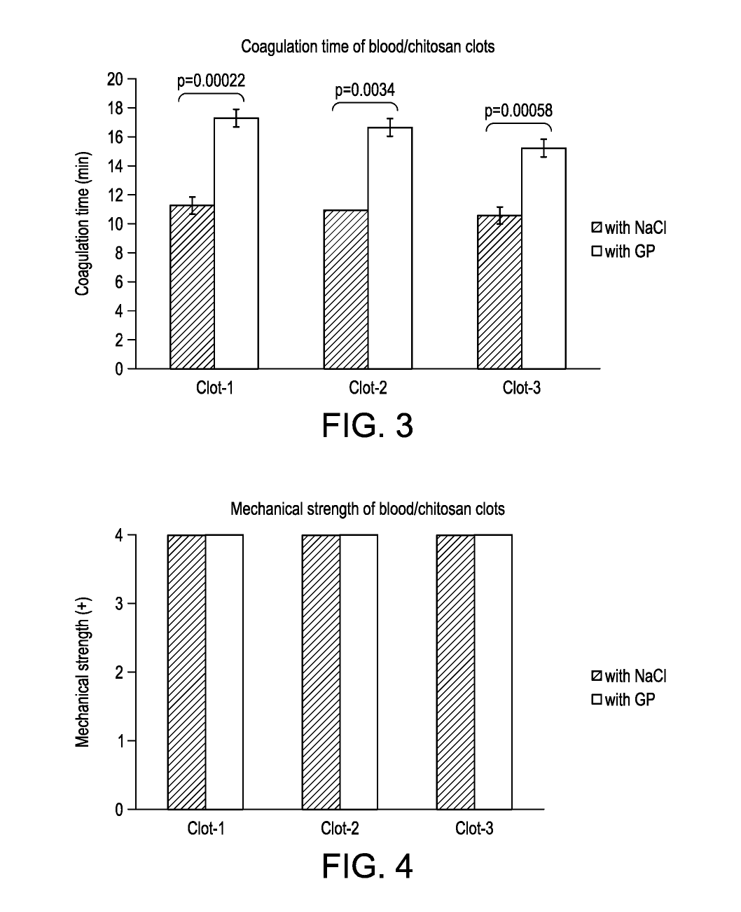

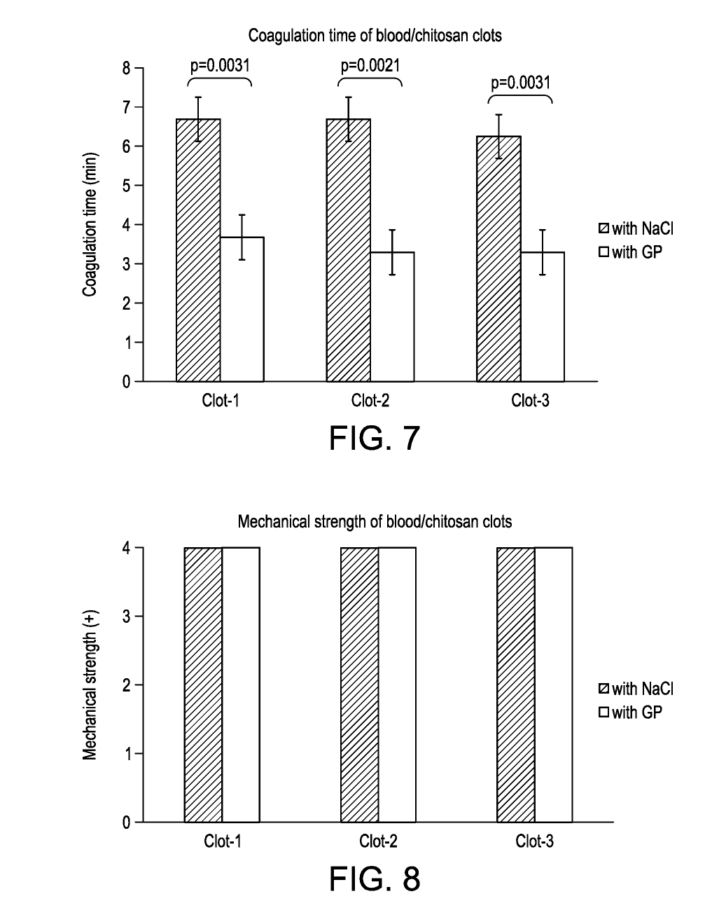

Another embodiment is shown in Example 2 where chitosans of different molecular weightsMₙ were used to prepare a novel formulation of physiological chitosan-HCl—NaCl mixtures (Table 8). Blood/chitosan-HCl—NaCl mixtures coagulated faster than blood/chitosan-HCl-βGP mixtures when the chitosan has a number average molecular weight Mₙ<232 kDa to create fast-coagulating blood/chitosan-HCl—NaCl implants with good mechanical strength for tissue repair (Tables 9-12 and FIGS. 3-6). Chitosans with number average molecular weights Mₙ>232 kDa were also used to prepare a novel formulation of physiological chitosan-HCl—NaCl mixtures that coagulated within 7 minutes to create fast-coagulating blood/chitosan implants with good mechanical strength (Tables 13-16 and FIGS. 7-10).

After 60 minutes, some serum exuded from the clots and the homogeneity evaluation results are shown in Table 17.

It is thus demonstrated herein that blood/chitosan-HCl—NaCl mixtures coagulated faster than blood/chitosan-GP mixtures when the chitosan had a molecular weight Mₙ<232 kDa to create blood/chitosan clots (implants) with good mechanical properties. Chitosans with a number average molecular weightMₙ>232 kDa were also used to prepare blood/chitosan-HCl—NaCl mixtures that coagulated to create blood/chitosan clots (implants) with good mechanical properties. Therefore, these solutions can be alternatives to the current chitosan-HCl-βGP solution known in the art with the advantage of solidifying faster.

The compositions described herein can be used to improve the repair and to regenerate cartilaginous tissues and other tissues including without limitation meniscus, ligament, tendon, bone, skin, comea, periodontal tissues, abscesses, resected tumors, and ulcers.

There is also contemplated herein the use of the polymer compositions described herein that can be placed or injected into a body site where the mixture aids the repair, regeneration, reconstruction or bulking of tissues. Repaired tissues include for example without limitation cartilage, meniscus, ligament, tendon, bone, skin, cornea, periodontal tissues, abscesses, resected tumors, and ulcers. The tissue that can be repaired or regenerated is for example without limitation cartilage, meniscus, ligament, tendon, bone, skin, cornea, periodontal tissue, abscesses, resected tumors, or ulcers. In some cases, the site of introduction in the body may be surgically prepared to remove abnormal tissues. Such procedure can be done by piercing, abrading or drilling into adjacent tissue regions or vascularized regions to create channels for the polymer composition to migrate into the site requiring repair.

The present disclosure will be more readily understood by referring to the following examples which are given to illustrate embodiments rather than to limit its scope.

### Example 1

### Formulation and Characterization of Fresh Blood/Physiological Chitosan Solution Mixtures (Rabbit Blood)

1—Preparation of Chitosan (1.62% w/w)-HCl (38 mM)-NaCl (160 mM) Solution; without NaOH, pH: 6.6; Total Volume: 10.0 ml

0.180 g of chitosan (degree of deacetylation or DDA=79% and number average weight or Mₙ75 kDa) was weighted in a 20 ml beaker. H₂Odd was added to the beaker until the weight of chitosan+H₂O=9.34 g. A magnetic stir bar was added into the beaker and the solution was stirred for about 10 minutes in order to hydrate the chitosan powder as much as possible. 0.38 ml of HCl 1 N (Sigma, Product No 318949) was added to the solution under moderate stirring. The beaker was covered with Parafilm™, and the solution was heated to about 60° C. for 2 hours, stirred overnight until completely dissolved. 0.32 ml of 5N NaCl (Sigma, Product No S-9888) solution was added into the beaker and mixed. The obtained pH of the chitosan solution was physiological at 6.5 and the osmolality was also physiological at 354 mOsm/kg (Table 1).

2—Preparation of Chltosan(1.62% w/w)-HCl (71 mM)-SGP (2.15%) Solution, pH: 6.6, Total Volume: 9.0 ml

0.162 g of chitosan (degree of deacetylation or DDA=79% and number average weight or Mₙ=75 kDa) was weighted in a 20 ml beaker. H₂Odd was added to the beaker until the weight of chitosan+H₂O=6.65 g. A magnetic stir bar was added into the beaker to stir the solution for about 10 minutes in order to hydrate the chitosan powder as much as possible. 0.55 ml of HCl 1 N (Sigma, Product No 318949) was added to the solution under moderate stirring. The beaker was covered with Parafilm™ and stirred overnight until completely dissolved. 1.8 ml of 10.75% pGP (Sigma, Product No G9891) in 50 mM HCl solution was added into the beaker and mixed (mix ratio of chitosan solution/PGP solution is 4:1). The obtained pH of the chitosan solution was physiological at 6.7 and the osmolality was also physiological at 334 mOsm/kg (Table 1).

TABLE 1
Composition and properties of chitosan solutions.

| Solution No and | C_{chitosan} | C_{HCl} | C_{NaCl} | C_{βGP} | | pH | Osmolality |
|---|---|---|---|---|---|---|---|
| type. | (% w/w) | (mM) | (mM) | (% w/w) | Precipitation | (Measured) | (mOsm/kg) |
| Chitosan- | 1.62 | 38 | 160 | — | no | 6.50 | 354 |
| HCl—NaCl | | | | | | | |
| pH 6.6 | | | | | | | |
| Chitosan- | 1.62 | 71 | — | 2.15 | no | 6.65 | 334 |
| HCl-βGP | | | | | | | |
| pH 6.6 | | | | | | | |

### 3—Drawing Blood

Blood was extracted from rabbits using sterile technique, starting by injecting 0.3 cc/kg Hypnorm® IM to the rabbits (for example 0.9 cc for a 3 kg rabbit). First, for each rabbit, ˜2 ml of blood was collected in a Vacutainer® tube containing EDTA (Fisher, BD, Product No 02-683-99A) to obtain CBC (complete blood count) and platelet count. Second, for each rabbit, ˜5 mL of blood was collected using a sterile 5 cc syringe (Fisher, BD, Product No 309604). Four rabbits were used in this experiment.

### 4—Preparing Blood/Physiological Chitosan Solution Mixtures, Measuring the Coagulation Time and Mechanical Strength of the Clots

To prepare blood/chitosan-HCl—NaCl (pH 6.6) clots-1, immediately following collection of whole blood, a 900 μl pipet of whole blood was added into a cryotube containing three 0.39 g stainless steel balls and 300 μl chitosan solution and mixed by hand shaking for 10 seconds. 250 μl was then transferred into 3 glass tubes at 37° C. with a 1 ml syringe to prepare 3 clots: the first clot was used to test coagulation time and fixed immediately after it coagulated; the second clot was used to test coagulation time and fixed at 60 minutes after mixing; the third clot was used to test coagulation time and mechanical strength after 60 minutes. This experiment was accomplished in triplicate for each rabbit. To prepare blood/chitosan-HCl-βGP (pH 6.6) clots-2, the same procedure was repeated as described hereinabove, but a chitosan-HCl-βGP (pH 6.6) solution was mixed with blood.

Coagulation was determined by visualization of the clot at 37° C. All the three glass tubes were used for testing coagulation time. The glass tubes were gently taken from the hot plate vertically every minute, slowly tilted, and the blood mixture was visualized at the bottom of tube. If the mixture was immobile and formed clot, it was coagulated; if the mixture was still mobile at the bottom of the tube, it was not coagulated yet. Mechanical strength was tested by putting the clot on the centre of the palm and pressing the clot with a finger until it was crushed. The resistance to compression, liquid expression and crushed appearance were also observed. The mechanical strength was scored with a 4 “+” system: “+” represents clot was easily broken and crushed appearance was multiple fragments (more than 5 fragments); “++” represents clot was relatively firm and crushed appearance was multiple fragments (3-5 fragments); “+++” represents clot was firm and elastic, crushed appearance was 2-3 fragments: “++++” represents clot was firm and elastic, crushed appearance was 2 fragments (sometimes still connected) or there was just a hole in the center of clot.

For Rabbit 259F (R1F), all the blood/chitosan mixtures coagulated within 10 minutes (from 7 minutes to 10 minutes) and formed firm elastic clots (see Table 2). The coagulation time of blood/chitosan-HCl—NaCl pH 6.6 mixtures (8 minutes) was significantly less than the coagulation time of blood/chitosan-HCl-PGP pH 6.6 mixtures (9.7 minutes) (FIG. 1).

TABLE 2
Coagulation time of blood/chitosan clots from rabbit R259F (R1F).

| | | Coagulation | Mean |
|---|---|---|---|
| | Sample | time | value |
| Mixture | number | (Min) | (Min |

| | | | | |
|---|---|---|---|---|
| Fresh | Mixture 1 | 1 | 8 | 8 |
| blood/chitosan- | | 2 | 8 | |
| HCl—NaCl pH 6.6 | | 3 | 8 | |
| (clot1) | Mixture 2 | 1 | 9 | |
| | | 2 | 9 | |
| | | 3 | 9 | |
| | Mixture 3 | 1 | 7 | |
| | | 2 | 7 | |
| | | 3 | 7 | |
| Fresh | Mixture 1 | 1 | 10 | 9.7 |
| blood/chitosan- | | 2 | 10 | |
| HCl- βGP pH 6.6 | | 3 | 10 | |
| (clot2) | Mixture 2 | 1 | 9 | |
| | | 2 | 9 | |
| | | 3 | 9 | |
| | Mixture 3 | 1 | 10 | |
| | | 2 | 10 | |
| | | 3 | 10 | |

For Rabbit 260F (R2F), all the blood/chitosan mixtures coagulated within 9 minutes and formed firm elastic clots (see Table 3 and FIG. 1).

TABLE 3
Coagulation time of blood/chitosan clots from rabbit R260F (R2F)

| | | | |
|---|---|---|---|
| | | Coagulation | Mean |
| | Sample | time | value |
| Mixture | number | (Min) | (Min) |

| | | | | |
|---|---|---|---|---|
| Fresh | Mixture 1 | 1 | 9 | 8.3 |
| blood/chitosan- | | 2 | 9 | |
| HCl—NaCl pH 6.6 | | 3 | 9 | |
| (clot1) | Mixture 2 | 1 | 8 | |
| | | 2 | 8 | |
| | | 3 | 8 | |
| | Mixture 3 | 1 | 8 | |
| | | 2 | 8 | |
| | | 3 | 8 | |
| Fresh | Mixture 1 | 1 | 9 | 8.7 |
| blood/chitosan- | | 2 | 9 | |
| HCl- βGP pH 6.6 | | 3 | 9 | |
| (clot2) | Mixture 2 | 1 | 9 | |
| | | 2 | 9 | |
| | | 3 | 9 | |
| | Mixture 3 | 1 | 8 | |
| | | 2 | 8 | |
| | | 3 | 8 | |

For Rabbit 261M (R3M), all the blood/chitosan mixtures coagulated within 15 minutes (from 6 minutes to 15 minutes) and formed firm elastic clots (see Table 4). The coagulation time of blood/chitosan-HCl—NaCl pH 6.6 mixtures (7.3 minutes) was significantly less than the coagulation time of blood/chitosan-HCl-βGP pH 6.6 mixtures (14.3 minutes) (FIG. 1).

TABLE 4
Coagulation time from rabbit R261M (R3M)

| | | | |
|---|---|---|---|
| | | Coagulation | Mean |
| | Sample | time | value |
| Mixture | number | (Min) | (Min) |

| | | | | |
|---|---|---|---|---|
| Fresh | Mixture 1 | 1 | 6 | 7.3 |
| blood/chitosan- | | 2 | 6 | |
| HCl—NaCl pH 6.6 | | 3 | 6 | |
| (clot1) | Mixture 2 | 1 | 8 | |
| | | 2 | 8 | |
| | | 3 | 8 | |
| | Mixture 3 | 1 | 8 | |
| | | 2 | 8 | |
| | | 3 | 8 | |
| Fresh | Mixture 1 | 1 | 14 | 14.3 |
| blood/chitosan- | | 2 | 14 | |
| HCl-βGP pH 6.6 | | 3 | 14 | |
| (clot2) | Mixture 2 | 1 | 15 | |
| | | 2 | 15 | |
| | | 3 | 15 | |
| | Mixture 3 | 1 | 14 | |
| | | 2 | 14 | |
| | | 3 | 14 | |

For Rabbit 262M (R3MR4M), all the blood/chitosan mixtures coagulated within 14 minutes (from 7 minutes to 14 minutes) and formed firm elastic clots (see Table 5). The coagulation time of blood/chitosan-HCl—NaCl pH 6.6 mixtures (8.3 minutes) was significantly less than the coagulation time of blood/chitosan-HCl-βGP pH 6.6 mixtures (12.7 minutes) (FIG. 1).

TABLE 5
Coagulation time from rabbit R262M (R4M)

| | | | |
|---|---|---|---|
| | | Coagulation | Mean |
| | Sample | time | value |
| Mixture | number | (Min) | (Min) |

| | | | | |
|---|---|---|---|---|
| Fresh | Mixture 1 | 1 | 7 | 8.3 |
| blood/chitosan- | | 2 | 7 | |
| HCl—NaCl pH 6.6 | | 3 | 7 | |
| (clot1) | Mixture 2 | 1 | 8 | |
| | | 2 | 8 | |
| | | 3 | 8 | |
| | Mixture 3 | 1 | 10 | |
| | | 2 | 10 | |
| | | 3 | 10 | |
| Fresh | Mixture 1 | 1 | 12 | 12.7 |
| blood/chitosan- | | 2 | 12 | |
| HCl-βGP pH 6.6 | | 3 | 12 | |
| (clot2) | Mixture 2 | 1 | 12 | |
| | | 2 | 12 | |
| | | 3 | 12 | |
| | Mixture 3 | 1 | 14 | |
| | | 2 | 14 | |
| | | 3 | 14 | |

The mechanical strength results (Table 6 and FIG. 2) demonstrated that all the fresh blood/chitosan clots were firm and elastic, the mechanical strengths of all the clots was scored as “++++”. After 60 minutes, there was some serum exuded from the clots (scored as ++ in most cases). Although the mechanical strength scores were identical for all clots, 9 of 12 βGP clots (clot2) had just a hole in the center while only 3 of 12 NaCl clots (clot1) had just a hole in the center, suggesting an improvement in mechanical strength.

TABLE 6
Mechanical strength test of blood/chitosan clots.

| Rabbit | Mixture | Sample | Resistance to | Liquid | Crushed |
|---|---|---|---|---|---|
| No. | component | No. | compression | expressed | appearance |
| R259F | Fresh | Clot1-1 | Firm and elastic | Some liquid exuded | Break into 2 fragments but |
| | blood/chitosan- | | ++++ | ++ | still connected |
| | HCl—NaCl pH 6.6 | Clot1-2 | Firm and elastic | Some liquid exuded | Break into 2 fragments but |
| | (clot1) | | ++++ | ++ | still connected |
| | | Clot1-3 | Firm and elastic | Some liquid exuded | Break into 2 fragments but |
| | | | ++++ | ++ | still connected |
| | Fresh | Clot2-1 | Firm and elastic | Some liquid exuded | Not broken but a hole in the |
| | blood/chitosan- | | ++++ | ++ | center of clot |
| | HCl- βGP pH 6.6 | Clot2-2 | Firm and elastic | Some liquid exuded | Not broken but a hole in the |
| | (clot2) | | ++++ | ++ | center of clot |
| | | Clot2-3 | Firm and elastic | Some liquid exuded | Break into 2 fragments but |
| | | | ++++ | ++ | still connected |
| R260F | Fresh | Clot1-1 | Firm and elastic | Some liquid exuded | Break into 2 fragments but |
| | blood/chitosan- | | ++++ | ++ | still connected |
| | HCl—NaCl pH 6.6 | Clot1-2 | Firm and elastic | Some liquid exuded | Break into 2 fragments but |
| | (clot1) | | ++++ | ++ | still connected |
| | | Clot1-3 | Firm and elastic | Some liquid exuded | Break into 2 fragments but |
| | | | ++++ | ++ | still connected |
| | Fresh | Clot2-1 | Firm and elastic | Some liquid exuded | Not broken but a hole in the |
| | blood/chitosan- | | ++++ | ++ | center of clot |
| | HCl- βGP pH 6.6 | Clot2-2 | Firm and elastic | Some liquid exuded | Not broken but a hole in the |
| | (clot2) | | ++++ | ++ | center of clot |
| | | Clot2-3 | Firm and elastic | Some liquid exuded | Not broken but a hole in the |
| | | | ++++ | ++ | center of clot |
| R261M | Fresh | Clot1-1 | Firm and elastic | Some liquid exuded | Not broken but a hole in the |
| | blood/chitosan- | | ++++ | ++ | center of clot |
| | HCl—NaCl pH 6.6 | Clot1-2 | Firm and elastic | Some liquid exuded | Not broken but a hole in the |
| | (clot1) | | ++++ | ++ | center of clot |
| | | Clot1-3 | Firm and elastic | Some liquid exuded | Break into 2 fragments but |
| | | | ++++ | ++ | still connected |
| | Fresh | Clot2-1 | Firm and elastic | Some liquid exuded | Not broken but a hole in the |
| | blood/chitosan- | | ++++ | ++ | center of clot |
| | HCl- βGP pH 6.6 | Clot2-2 | Firm and elastic | Some liquid exuded | Not broken but a hole in the |
| | (clot2) | | ++++ | ++ | center of clot |
| | | Clot2-3 | Firm and elastic | Some liquid exuded | Not broken but a hole in the |
| | | | ++++ | ++ | center of clot |
| R262M | Fresh | Clot1-1 | Firm and elastic | Some liquid exuded | Break into 2 fragments but |
| | blood/chitosan- | | ++++ | ++ | still connected |
| | HCl—NaCl pH 6.6 | Clot1-2 | Firm and elastic | Some liquid exuded | Not broken but a hole in the |
| | (clot1) | | ++++ | +++ | center of clot |
| | | Clot1-3 | Firm and elastic | Some liquid exuded | Break into 2 fragments but |
| | | | ++++ | + | still connected |
| | Fresh | Clot2-1 | Firm and elastic | Some liquid exuded | Not broken but a hole in the |
| | blood/chitosan- | | ++++ | ++ | center of clot |
| | HCl- βGP pH 6.6 | Clot2-2 | Firm and elastic | Some liquid exuded | Not broken but a hole in the |
| | (clot2) | | ++++ | ++ | center of clot |
| | | Clot2-3 | Firm and elastic | Some liquid exuded | Break into 2 fragments but |
| | | | ++++ | ++ | still connected |

### 5—Histological Treatment and Homogeneity Evaluation of Blood/Chitosan Clots

Fixed clots were sectioned in two parts. One part was cryoprotected with sucrose, infiltrated with OCT, cryosectioned, stained with Safranin O/Fast Green and observed by optical microscopy. One part was stored at 4° C. until further use. All the photos were taken from the blood/chitosan clot samples fixed 60 minutes after the clots were prepared; two photos taken with 5× and 40× objectives from different regions of each sample were used for homogeneity evaluation. Each specimen was observed under microscopy with special emphasis on: presence of bubbles or cracks; presence and distribution of precipitates of chitosan described as: large aggregates or small aggregates; chitosan distribution and whether or not they are homogeneously dispersed across the section; erythrocyte morphology in terms of discoid, shrunken, swollen or chaining.

Histology showed that the homogeneity of clots prepared with βGP was better (8 of 12 samples scored as “+” and none scored as “−”) than the homogeneity of the clots prepared with NaCl (3 of 11 samples scored as “+” and 3 of 11 samples scored as “−”) (Table 7).

TABLE 7
Homogeneity of blood/chitosan clots.

| Clot-1 | | Clot-2 | |
|---|---|---|---|
| sample | Homogeneity of | sample | Homogeneity of |
| (with NaCl) | clot-1 | (with βGP) | clot-2 |
| FB 1-1-R259F | + | FB 2-1-R259F | + |
| FB 1-2-R259F | + | FB 2-2-R259F | + |
| FB 1-3-R259F | − | FB 2-3-R259F | ± |
| FB 1-1-R260F | + | FB 2-1-R260F | + |
| FB 1-2-R260F | ± | FB 2-2-R260F | + |
| FB 1-3-R260F | − | FB 2-3-R260F | + |
| FB 1-1-R261M | ± | FB 2-1-R261M | ± |
| FB 1-2-R261M | − | FB 2-2-R261M | + |
| FB 1-3-R261M | * | FB 2-3-R261M | + |
| FB 1-1-R262M | ± | FB 2-1-R262M | + |
| FB 1-2-R262M | ± | FB 2-2-R262M | ± |
| FB 1-3-R262M | + | FB 2-3-R262M | ± |

* data not available

### Example 2

### Formulation and Characterization of Fresh Blood/Chitosan Clots by Using Chitosan Solutions with Different Molecular Weight (Rabbit Blood)

1—Preparation of Chitosan (1.62% w/w)-HCl (38 mM)-NaCl (160 mM) Solution; without NaOH, pH: 6.6; Total Volume: 10.0 ml

The chitosan-HCl—NaCl solutions were prepared as described previously with chitosans of different Mₙ and DDA (lot No. AS-144-02-A: Mₙ 2.7 kDa and DDA 98%; lot No. CH10075: Mₙ 75 kDa and DDA 79%; lot No. CH0100702B: Mₙ 232 kDa, and DDA 81%; lot No. CH0050602A: Mₙ 298 kDa and DDA 76%). The pH of the chitosan solutions were physiological (6.2 to 6.7) and the osmolality was also physiological (326 to 356 mOsm/kg) (see Table 8).

2—Preparation of Chitosan (1.62% w/w)-HCl (71 mM)-βGP (2.15%) Solution, pH: 6.6, Total Volume: 9.0 ml

The chitosan-HCl—NaCl solutions were prepared as described previously with chitosans of different Mₙ and DDA (lot No. AS-144-02-A: Mₙ 2.7 kDa and DDA 98%; lot No. CH10075: Mₙ 75 kDa and DDA 79%; lot No. CH0100702B: Mₙ 232 kDa, and DDA 81%; lot No. CH0050602A: Mₙ 298 kDa and DDA 76%). The pH of the chitosan solutions were physiological at 6.7 and the osmolality was also physiological (340 to 345 mOsm/kg) (Table 8).

TABLE 8
Composition and properties of chitosan solutions

| | C_{chitosan} | C_{HCl} | C_{NaCl} | C_{βGP} | | pH | Osmolarity |
|---|---|---|---|---|---|---|---|
| Solution No and type. | (% w/w) | (mM) | (mM) | (% w/w) | Precipitation | (Measured) | (mOsm) |
| Chitosan-HCl—NaCl | 1.62 | 55 | 160 | no | no | 6.19 | 356 |
| pH 6.6 (AS-144-02-A) | | | | | | | |
| Chitosan-HCl-βGP | 1.62 | 77 | No | 2.15 | no | 6.69 | 345 |
| pH 6.6 (AS-144-02-A) | | | | | | | |
| Chitosan-HCl—NaCl | 1.62 | 38 | 160 | no | no | 6.62 | 326 |
| pH 6.6 (CH10075) | | | | | | | |
| Chitosan-HCl-βGP | 1.62 | 71 | No | 2.15 | no | 6.67 | 344 |
| pH 6.6 (CH10075) | | | | | | | |
| Chitosan-HCl—NaCl | 1.62 | 38 | 160 | no | no | 6.65 | 339 |
| pH 6.6 (CH0100702B) | | | | | | | |
| Chitosan-HCl-βGP | 1.62 | 71 | No | 2.15 | no | 6.71 | 337 |
| pH 6.6 (CH0100702B) | | | | | | | |
| Chitosan-HCl—NaCl | 1.62 | 38 | 160 | no | no | 6.58 | 332 |
| pH 6.6 (CH0050602A) | | | | | | | |
| Chitosan-HCl-βGP | 1.62 | 71 | No | 2.15 | no | 6.68 | 340 |
| pH 6.6 (CH0050602A) | | | | | | | |

### 3—Drawing Blood

Blood was extracted from rabbits using sterile technique, as described previously. Four rabbits were used in this experiment.

### 4—Preparing Blood/Physiological Chitosan Solution Mixtures, Measuring the Coagulation Time and Mechanical Strength of the Clots

Rabbit whole blood/chitosan-HCl—NaCl mixtures and rabbit whole blood/chitosan-HCl-βGP mixtures were prepared at a mix ratio of 3:1 v/v as described previously. This experiment was accomplished in triplicate for each rabbit. Coagulation of the clot was determined by visualization of the clot at 37° C. as described previously. Mechanical strength was tested as described previously.

For chitosan lot No. AS-144-02-A (Mₙ of 2.7 kDa and 98% DDA), all the blood/chitosan mixtures coagulated within 18 minutes (from 10 minutes to 18 minutes) and formed firm elastic clots (see Table 9). The coagulation time of blood/chitosan-HCl—NaCl pH 6.6 mixtures was significantly less than the coagulation time of blood/chitosan-HCl-PGP pH 6.6 mixtures (FIG. 3). The mechanical strength test results showed that all the clots were firm and elastic, the mechanical strengths of all the clots was scored as “++++” and the clots did not retract significantly (see Table 10 and FIG. 4).

TABLE 9
Coagulation time of fresh blood/chitosan
mixtures (Lot No. AS-144-02-A)

| | Concentration of | | Coagulation | Mean | |
|---|---|---|---|---|---|
| | chitosan solution | | time | value | |
| Mixture | and mixing ratio | | (Min) | (Min) | Comments |
| Fresh blood/ | 1.62% and 3:1 | 1 | 12 | 11.3 | All the mix- |
| Chitosan- | (clot1-1-1) | 2 | 11 | | ture samples |
| HCl—NaCl | | 3 | 11 | | coagulated |
| | 1.62% and 3:1 | 1 | 11 | 11 | within 12 |
| | (clot1-1-2) | 2 | 11 | | minutes (from |
| | | 3 | 11 | | 10 minutes to |
| | 1.62% and 3:1 | 1 | 11 | 10.7 | 12 minutes). |
| | (clot1-1-3) | 2 | 10 | | |
| | | 3 | 11 | | |
| Fresh blood/ | 1.62% and 3:1 | 1 | 18 | 17.3 | All the mix- |
| Chitosan- | (clot1-2-1) | 2 | 17 | | ture samples |
| HCl- βGP | | 3 | 17 | | coagulated |
| | 1.62% and 3:1 | 1 | 17 | 16.7 | within 18 |
| | (clot1-2-2) | 2 | 16 | | minutes (from |
| | | 3 | 17 | | 15 minutes to |
| | 1.62% and 3:1 | 1 | 15 | 15.3 | 18 minutes). |
| | (clot1-2-3) | 2 | 15 | | |
| | | 3 | 16 | | |

TABLE 10
Mechanical strength test of fresh blood/chitosan
clots (Lot No. AS-144-02-A)

| | Resistance to | Liquid | Crushed |
|---|---|---|---|
| Sample | compression | expressed | appearance |
| Fresh blood/ | Firm and elastic | Almost no liquid | 2 fragments |
| Chitosan- | ++++ | expressed | |
| HCl—NaCl | | + | |
| clot1-1-1 | | | |
| (1.62%, 3:1) | | | |
| Fresh blood/ | Firm and elastic | Almost no liquid | 2 fragments |
| Chitosan- | ++++ | expressed | |
| HCl—NaCl | | + | |
| clot1-1-2 | | | |
| (1.62%, 3:1) | | | |
| Fresh blood/ | Firm and elastic | A few liquid | 2 fragments |
| Chitosan- | ++++ | expressed | |
| HCl—NaCl | | ++ | |
| clot1-1-3 | | | |
| (1.62%, 3:1) | | | |
| Fresh blood/ | Firm and elastic | Almost no liquid | 2 fragments |
| Chitosan- | ++++ | expressed | |
| HCl- βGP | | + | |
| clot1-2-1 | | | |
| (1.62%, 3:1) | | | |
| Fresh blood/ | Firm and elastic | Almost no liquid | 2 fragments but |
| Chitosan- | ++++ | expressed | still connected |
| HCl- βGP | | + | |
| clot1-2-2 | | | |
| (1.62%, 3:1) | | | |
| Fresh blood/ | Firm and elastic | Almost no liquid | 2 fragments |
| Chitosan- | ++++ | expressed | |
| HCl- βGP | | + | |
| clot1-2-3 | | | |
| (1.62%, 3:1) | | | |

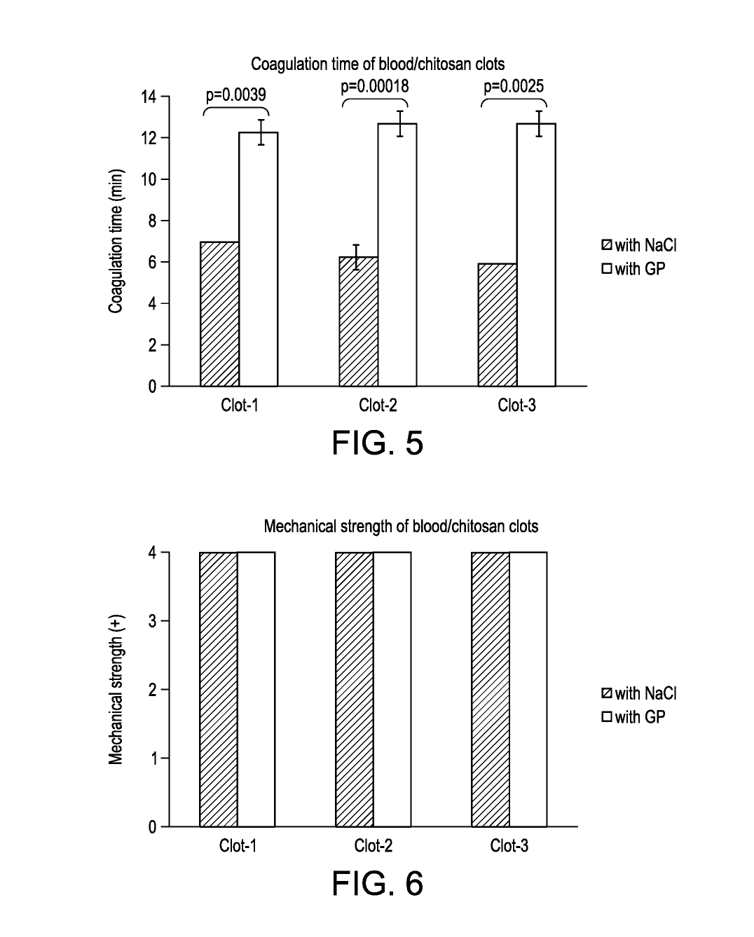

For chitosan lot No. CH10075 (Mₙ of 75 kDa and 79% DDA), all the blood/chitosan mixtures coagulated within 13 minutes (from 6 minutes to 13 minutes) and formed firm elastic clots (Table 11). The coagulation time of blood/chitosan-HCl—NaCl pH 6.6 mixtures was significantly less than the coagulation time of blood/chitosan-HCl-pGP pH 6.6 mixtures (FIG. 5). The mechanical strength test results showed that all the clots were firm and elastic, the mechanical strengths of all the clots was scored as “++++” and the clots did not retract significantly (see Table 12 and FIG. 6).

TABLE 11
Coagulation time of fresh blood/chitosan
mixtures (Lot No. CH10075)

| | Concentration of | | Coagulation | Mean | |
|---|---|---|---|---|---|
| | chitosan solution | | time | value | |
| Mixture | and mixing ratio | | (Min) | (Min) | Comments |
| Fresh blood/ | 1.62% and 3:1 | 1 | 7 | 7 | All the mix- |
| Chitosan- | (clot2-1-1) | 2 | 7 | | ture samples |
| HCl—NaCl | | 3 | 7 | | coagulated |
| | 1.62% and 3:1 | 1 | 6 | 6.3 | within 7 |
| | (clot2-1-2) | 2 | 7 | | minutes (from |
| | | 3 | 6 | | 6 minutes to |
| | 1.62% and 3:1 | 1 | 6 | 6 | 7 minutes). |
| | (clot2-1-3) | 2 | 6 | | |
| | | 3 | 6 | | |
| Fresh blood/ | 1.62% and 3:1 | 1 | 12 | 12.3 | All the mix- |
| Chitosan- | (clot2-2-1) | 2 | 12 | | ture samples |
| HCl- βGP | | 3 | 13 | | coagulated |
| | 1.62% and 3:1 | 1 | 13 | 12.7 | within 13 |
| | (clot2-2-2) | 2 | 13 | | minutes (from |
| | | 3 | 12 | | 12 minutes to |
| | 1.62% and 3:1 | 1 | 13 | 12.7 | 13 minutes). |
| | (clot2-2-3) | 2 | 12 | | |
| | | 3 | 13 | | |

TABLE 12
Mechanical test of fresh blood/chitosan clots (Lot No. CH10075)

| | Resistance to | Liquid | Crushed |
|---|---|---|---|
| Sample | compression | expressed | appearance |
| Fresh blood/ | Firm and elastic | Almost no liquid | 2 fragments but |
| Chitosan- | ++++ | expressed | still connected |
| HCl—NaCl | | + | |
| clot2-1-1 | | | |
| (1.62%, 3:1) | | | |
| Fresh blood/ | Firm and elastic | Almost no liquid | 2 fragments |
| Chitosan- | ++++ | expressed | |
| HCl—NaCl | | + | |
| clot2-1-2 | | | |
| (1.62%, 3:1) | | | |
| Fresh blood/ | Firm and elastic | Almost no liquid | 2 fragments |
| Chitosan- | ++++ | expressed | |
| HCl—NaCl | | + | |
| clot2-1-3 | | | |
| (1.62%, 3:1) | | | |
| Fresh blood/ | Firm and elastic | Almost no liquid | 2 fragments but |
| Chitosan- | ++++ | expressed | still connected |
| HCl- βGP | | + | |
| clot2-2-1 | | | |
| (1.62%, 3:1) | | | |
| Fresh blood/ | Firm and elastic | Almost no liquid | 2 fragments |
| Chitosan- | ++++ | expressed | |
| HCl- βGP | | + | |
| clot2-2-2 | | | |
| (1.62%, 3:1) | | | |
| Fresh blood/ | Firm and elastic | Almost no liquid | 2 fragments but |
| Chitosan- | ++++ | expressed | still connected |
| HCl- βGP | | + | |
| clot2-2-3 | | | |
| (1.62%, 3:1) | | | |

For chitosan lot No. CH0100702B (Mₙ of 232 kDa and 81% DDA), all the blood/chitosan mixtures coagulated within 7 minutes (from 3 minutes to 7 minutes) and formed firm elastic clots (see Table 13 and FIG. 7). The mechanical strength test results showed that all the clots were firm and elastic, the mechanical strengths of all the clots was scored as “++++” and the clots did not retract significantly (see Table 14 and FIG. 8).

TABLE 13
Coagulation time of fresh blood/chitosan
mixture (Lot No. CH0100702B)

| | Concentration of | | Coagulation | Mean | |
|---|---|---|---|---|---|
| | chitosan solution | | time | value | |
| Mixture | and mixing ratio | | (Min) | (Min) | Comments |
| Fresh blood/ | 1.62% and 3:1 | 1 | 7 | 6.7 | All the mix- |
| Chitosan- | (clot3-1-1) | 2 | 7 | | ture samples |
| HCl—NaCl | | 3 | 6 | | coagulated |
| | 1.62% and 3:1 | 1 | 7 | 6.7 | within 7 |
| | (clot3-1-2) | 2 | 7 | | minutes (from |
| | | 3 | 6 | | 6 minutes to |
| | 1.62% and 3:1 | 1 | 7 | 6.3 | 7 minutes). |
| | (clot3-1-3) | 2 | 6 | | |
| | | 3 | 6 | | |
| Fresh blood/ | 1.62% and 3:1 | 1 | 3 | 3.7 | All the mix- |
| Chitosan- | (clot3-2-1) | 2 | 4 | | ture samples |
| HCl- βGP | | 3 | 4 | | coagulated |
| | 1.62% and 3:1 | 1 | 4 | 3.3 | within 4 |
| | (clot3-2-2) | 2 | 3 | | minutes (from |
| | | 3 | 3 | | 3 minutes to |
| | 1.62% and 3:1 | 1 | 3 | 3.3 | 4 minutes). |
| | (clot3-2-3) | 2 | 3 | | |
| | | 3 | 4 | | |

TABLE 14
Mechanical test of fresh blood/chitosan
clots (Lot No. CH0100702B)

| | Resistance to | Liquid | Crushed |
|---|---|---|---|
| Sample | compression | expressed | appearance |
| Fresh blood/ | Firm and elastic | Almost no liquid | 2 fragments |
| Chitosan- | ++++ | expressed | |
| HCl—NaCl | | + | |
| clot3-1-1 | | | |
| (1.62%, 3:1) | | | |
| Fresh blood/ | Firm and elastic | Almost no liquid | 2 fragments |
| Chitosan- | ++++ | expressed | |
| HCl—NaCl | | ++ | |
| clot3-1-2 | | | |
| (1.62%, 3:1) | | | |
| Fresh blood/ | Firm and elastic | Almost no liquid | 2 fragments but |
| Chitosan- | ++++ | expressed | still connected |
| HCl—NaCl | | + | |
| clot3-1-3 | | | |
| (1.62%, 3:1) | | | |
| Fresh blood/ | Firm and elastic | Almost no liquid | 2 fragments but |
| Chitosan- | ++++ | expressed | still connected |
| HCl- βGP | | ++ | |
| clot3-2-1 | | | |
| (1.62%, 3:1) | | | |
| Fresh blood/ | Firm and elastic | Almost no liquid | 2 fragments but |
| Chitosan- | ++++ | expressed | still connected |
| HCl- βGP | | + | |
| clot3-2-2 | | | |
| (1.62%, 3:1) | | | |
| Fresh blood/ | Firm and elastic | Almost no liquid | 2 fragments but |
| Chitosan- | ++++ | expressed | still connected |
| HCl- βGP | | + | |
| clot3-2-3 | | | |
| (1.62%, 3:1) | | | |

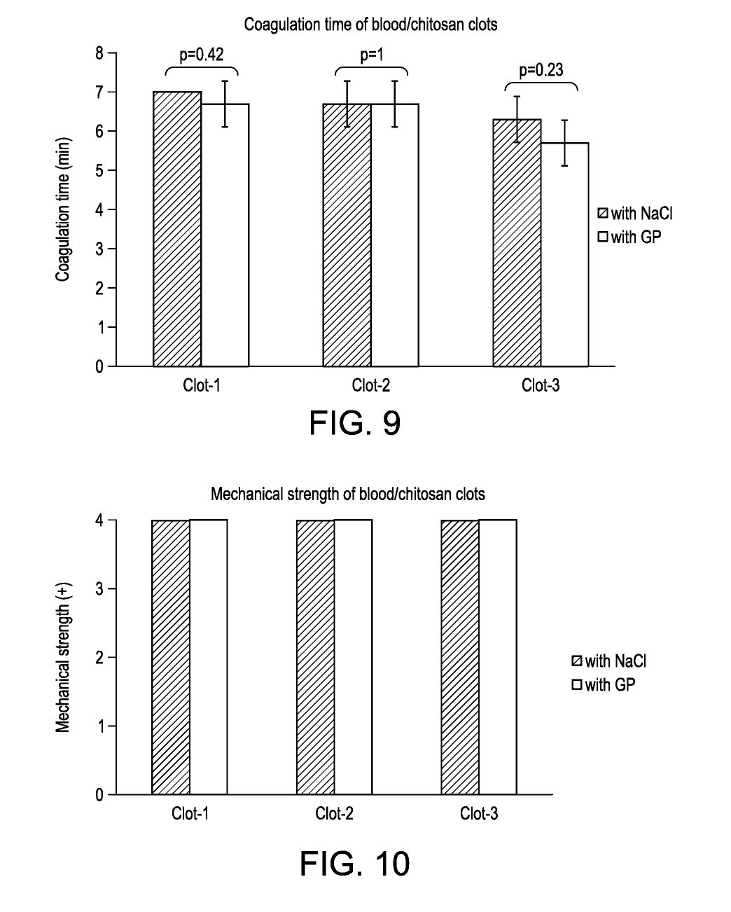

For chitosan lot No. CH0050602A (Mₙ of 298 kDa, 76% DDA), all the blood/chitosan mixtures coagulated within 7 minutes (from 5 minutes to 7 minutes) and formed firm elastic clots (see Table 15 and FIG. 9). The mechanical strength test results showed that all the clots were firm and elastic, the mechanical strengths of all the clots was scored as “++++” and the clots did not retract significantly (see Table 16 and FIG. 10).

TABLE 15
Coagulation time of fresh blood/chitosan
mixture (Lot No. CH0050602A)

| | Concentration of | | Coagulation | Mean | |
|---|---|---|---|---|---|
| | chitosan solution | | time | value | |
| Mixture | and mixing ratio | | (Min) | (Min) | Comments |
| Fresh blood/ | 1.62% and 3:1 | 1 | 7 | 7 | All the mix- |
| Chitosan- | (clot4-1-1) | 2 | 7 | | ture samples |
| HCl—NaCl | | 3 | 7 | | coagulated |
| | 1.62% and 3:1 | 1 | 7 | 6.7 | within 7 |
| | (clot4-1-2) | 2 | 7 | | minutes (from |
| | | 3 | 6 | | 6 minutes to |
| | 1.62% and 3:1 | 1 | 7 | 6.3 | 7 minutes). |
| | (clot4-1-3) | 2 | 6 | | |
| | | 3 | 6 | | |
| Fresh blood/ | 1.62% and 3:1 | 1 | 7 | 6.7 | All the mix- |
| Chitosan- | (clot4-2-1) | 2 | 6 | | ture samples |
| HCl- βGP | | 3 | 7 | | coagulated |
| | 1.62% and 3:1 | 1 | 7 | 6.7 | within 7 |
| | (clot4-2-2) | 2 | 7 | | minutes (from |
| | | 3 | 6 | | 5 minutes to |
| | 1.62% and 3:1 | 1 | 6 | 5.7 | 7 minutes). |
| | (clot4-2-3) | 2 | 6 | | |
| | | 3 | 5 | | |

TABLE 16
Mechanical test of fresh blood/chitosan
clots (Lot No. CH0050602A)

| | Resistance to | Liquid | Crushed |
|---|---|---|---|
| Sample | compression | expressed | appearance |
| Fresh blood/ | Firm and elastic | Almost no liquid | 2 fragments |
| Chitosan- | ++++ | expressed | |
| HCl—NaCl | | + | |
| clot4-1-1 | | | |
| (1.62%, 3:1) | | | |
| Fresh blood/ | Firm and elastic | Almost no liquid | 2 fragments but |
| Chitosan- | ++++ | expressed | still connected |
| HCl—NaCl | | + | |
| clot4-1-2 | | | |
| (1.62%, 3:1) | | | |
| Fresh blood/ | Firm and elastic | Almost no liquid | 2 fragments but |
| Chitosan- | ++++ | expressed | still connected |
| HCl—NaCl | | + | |
| clot4-1-3 | | | |
| (1.62%, 3:1) | | | |
| Fresh blood/ | Firm and elastic | Almost no liquid | Hole in center |
| Chitosan- | ++++ | expressed | |
| HCl- βGP | | + | |
| clot4-2-1 | | | |
| (1.62%, 3:1) | | | |
| Fresh blood/ | Firm and elastic | Almost no liquid | 2 fragments but |
| Chitosan- | ++++ | expressed | still connected |
| HCl- βGP | | + | |
| clot4-2-2 | | | |
| (1.62%, 3:1) | | | |
| Fresh blood/- | Firm and elastic | Almost no liquid | 2 fragments but |
| Chitosan- | ++++ | expressed | still connected |
| HCl- βGP | | + | |
| clot4-2-3 | | | |
| (1.62%, 3:1) | | | |

### 5—Histological Treatment and Homogeneity Evaluation of Blood/Chitosan Clots

Histological treatment and homogeneity evaluation was performed as described hereinabove.

Histology showed that the homogeneity of clots prepared with RGP was better (9 of 12 samples scored as “+” and none scored as “−”) than the homogeneity of the clots prepared with NaCl (2 of 12 samples scored as “+” and 7 of 12 samples scored as “−”) (see Table 17).

TABLE 17
Homogeneity of blood/chitosan clots

| | Homogeneity | | Homogeneity |
|---|---|---|---|
| Clot samples | of clots | Clot samples | of clots |
| (with NaCl) | (with NaCl) | (with βGP) | (with βGP) |
| blood 1-1-1 | + (between ± | blood 1-2-1 | ± |
| (AS-144-02-A) | and +) | (AS-144-02-A) | |
| blood 1-1-2 | ± (between ± | blood 1-2-2 | + (between ± |
| (AS-144-02-A) | and +) | (AS-144-02-A) | and +) |
| blood 1-1-3 | ± (between ± | blood 1-2-3 | + (between ± |
| (AS-144-02-A) | and +) | (AS-144-02-A) | and +) |
| blood 2-1-1 | − | blood 2-2-1 | + |
| (CH10075) | | (CH10075) | |
| blood 2-1-2 | − | blood 2-2-2 | + (between ± |
| (CH10075) | | (CH10075) | and +) |
| blood 2-1-3 | − | blood 2-2-3 | + |
| (CH10075) | | (CH10075) | |
| blood 3-1-1 | + | blood 3-2-1 | + |
| (CH0100702B) | | (CH0100702B) | |
| blood 3-1-2 | + | blood 3-2-2 | + |
| (CH0100702B) | | (CH0100702B) | |
| blood 3-1-3 | − | blood 3-2-3 | + |
| (CH0100702B) | | (CH0100702B) | |
| blood 4-1-1 | − | blood 4-2-1 | + |
| (CH0050602A) | | (CH0050602A) | |
| blood 4-1-2 | − | blood 4-2-2 | + |
| (CH0050602A) | | (CH0050602A) | |
| blood 4-1-3 | − | blood 4-2-3 | + |
| (CH0050602A) | | (CH0050602A) | |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method for repairing a tissue in a subject in need thereof, said method comprising the step of introducing into said tissue a polymer composition, wherein said polymer composition comprises a blood component, a polymer and at least one inorganic salt, wherein said polymer composition is absent a glycerol-phosphate such that the polymer composition adheres to the tissue and promotes cell proliferation for repairing the tissue.

2. The method of claim 1 wherein said composition has a mean coagulation time of less than twelve minutes.

3. The method of claim 1, wherein said tissue is selected from the group consisting of cartilage, meniscus, ligament, tendon, bone, skin, cornea, periodontal tissues, maxillofacial tissues, temporomandibular tissues, abscesses, resected tumors and ulcers.

4. The method of claim 1 wherein said polymer composition is suitable for use as a solidifying implant for repairing the tissue.

5. The method of claim 1 wherein the polymer composition further comprises a mineral acid.

6. The method of claim 1 wherein the polymer is chitosan.

7. The method of claim 1, wherein the at least one inorganic salt is a sodium salt, chloride salt, potassium salt, calcium salt, magnesium salt, phosphate salt, sulfate salt or carboxylate salt.

8. The method of claim 1, wherein the at least one inorganic salt is NaCl.

9. The method of claim 1, wherein the blood component is whole blood.

10. The method of claim 1, wherein the composition is a gel.

11. The method of claim 1, wherein the blood component is coagulated.

12. The method of claim 1, wherein the polymer composition has a pH between 6.0 and 7.8.

13. The method of claim 12, wherein the polymer composition has a pH between 6.2 and 6.7.

14. The method of claim 13, wherein the polymer composition has a pH of 6.6.

15. The method of claim 1, wherein the composition has an osmolality between 200 mOsm/kg and 600 mOsm/kg.

16. The method of claim 15, wherein the osmolality is between 326 mOsm/kg and 356 mOsm/kg.

17. The method of claim 15, wherein the osmolality is of 354 mOsm/kg.

18. The method of claim 1, wherein the polymer is chitosan with a degree of deacetylation (DDA) between 20% to 100%.

19. The method of claim 18, wherein the DDA is between 76% and 98%.

20. The method of claim 1, wherein the polymer is chitosan with a number average molecular weight (Mn) between 1 kDa to 10 MDa.

21. The method of claim 19, wherein the Mn is between 2.7 kDa and 298 kDa.

22. The method of claim 5, wherein the mineral acid is hydrochloric acid.

23. The method of claim 1, wherein the blood component to polymer has a ratio of 3:1 v/v.

24. A method of preparing a polymer composition for repairing a tissue in a subject, said method comprising the steps of: a) dissolving chitosan in HCl to provide a chitosan-HCl mixture; b) adding a NaCl solution to the chitosan-HCl mixture to provide a chitosan-HCl—NaCl mixture; and c) admixing at least one blood component to the chitosan-HCl—NaCl mixture to provide the polymer composition.
